(19) United States
(12) Patent Application Publication
(10) Pub. No.: US 2024/0284012 A1
(43) Pub. Date: Aug. 22, 2024

(54) VIDEO PLAYBACK SYSTEM

(71) Applicant: TOSHIBA TEC KABUSHIKI KAISHA, Tokyo (JP)

(72) Inventor: Tatsuya Kaneko, Mishima Shizuoka (JP)

(21) Appl. No.: 18/512,074

(22) Filed: Nov. 17, 2023

(30) Foreign Application Priority Data
Feb. 22, 2023 (JP) 2023-026492

(51) Int. Cl. H04N 21/488; G10L 15/26

(52) U.S. Cl. CPC H04N 21/4884; G10L 15/26

(57) ABSTRACT

An after-the-fact check of a face-to-face interaction between a plurality of people with a conversation. A video playback system includes a video acquisition unit, a video memory unit, a text acquisition unit, a text memory unit, a video display unit, and a text display unit. The video memory unit stores video data representing a video captured over an image capture area and acquired by the video acquisition unit. The text memory unit stores text data generated by recognizing a voice collected around the image capture area and acquired by the text acquisition unit. The text display unit selects text data formed by recognizing a voice collected around the image capture area at an image capture timing of the video displayed by the video display unit, from among the stored text data, and displays a text represented by the text data.

### CROSS-REFERENCE TO RELATED APPLICATION

This application is based upon and claims the benefit of priority from Japanese Patent Application No. 2023-026492, filed on Feb. 22, 2023, the entire contents of which are incorporated herein by reference.

### FIELD

Embodiments described herein relate generally to a video playback system and video processing methods.

### BACKGROUND

In a case where a plurality of people interact with each other face to face, as in a customer service provided by a store clerk with a conversation at a store counter or the like, it is already a common practice to record the interaction as a video file with voice.

However, in the case of checking a part where a certain utterance is made from the video file with voice, the checker can only find out the part while checking the image and the voice. This causes a very heavy burden on the checker.

In view of such circumstances, it is desired to facilitate an after-the-fact of check a face-to-face interaction between a plurality of people with a conversation.

### DESCRIPTION OF THE DRAWINGS

FIG. 1 is a block diagram showing a schematic configuration of a recording system according to an embodiment, and a main circuit configuration of a recording and playback device and a counter terminal included in the recording system.

FIG. 2 schematically shows a data structure of service response management data in FIG. 1.

FIG. 3 is a flowchart of service response registration processing.

FIG. 4 shows an example of a registration screen.

FIG. 5 is a flowchart of recording and playback processing.

FIG. 6 shows an example of the registration screen.

FIG. 7 shows an example of the registration screen.

FIG. 8 shows an example of the registration screen.

FIG. 9 is a flowchart of voice recognition processing.

FIG. 10 shows an example of a start instruction screen.

FIG. 11 shows an example of a stop instruction screen.

FIG. 12 is a flowchart of search processing.

FIG. 13 is a flowchart of the search processing.

FIG. 14 is a flowchart of the search processing.

FIG. 15 shows an example of a search screen.

FIG. 16 shows an example of a playback screen.

FIG. 17 shows an example of a screen where an editing window is displayed as a popup.

### DETAILED DESCRIPTION

An embodiment described herein is to provide a video playback system that can facilitate an after-the-fact check of a face-to-face interaction between a plurality of people with a conversation.

In general, according to one embodiment, a video playback system includes a video acquisition unit, a video memory unit, a text acquisition unit, a text memory unit, a video display unit, and a text display unit. The video acquisition unit acquires video data representing a video captured over a predetermined image capture area. The video memory unit stores the video data acquired by the video acquisition unit. The text acquisition unit acquires text data generated by recognizing a voice collected around the image capture area. The text memory unit stores the text data acquired by the text acquisition unit. The video display unit displays the video represented by the video data stored in the video memory unit. The text display unit selects text data generated by recognizing a voice collected around the image capture area at an image capture timing of the video displayed by the video display unit, from among the text data stored in the text memory unit, and displays a text represented by the selected text data. According to another embodiment, a video processing method involves acquiring video data representing a video captured over a predetermined image capture area; storing the video data acquired; acquiring text data generated by recognizing a voice collected around the image capture area; storing the text data acquired; displaying the video represented by the video data stored; and selecting text data generated by recognizing a voice collected around the image capture area at an image capture timing of the video displayed, from among the text data stored, and displaying a text represented by the selected text data.

An example of embodiment will now be described, using the drawings. In this embodiment, an example where a video playback system is applied to a recording system that records a service response by a store clerk at a counter to a patient receiving a prepared medicine from a pharmacy (hereinafter referred to as a service response to patient) is described. The patient receiving a prepared medicine from the pharmacy is a customer of the pharmacy.

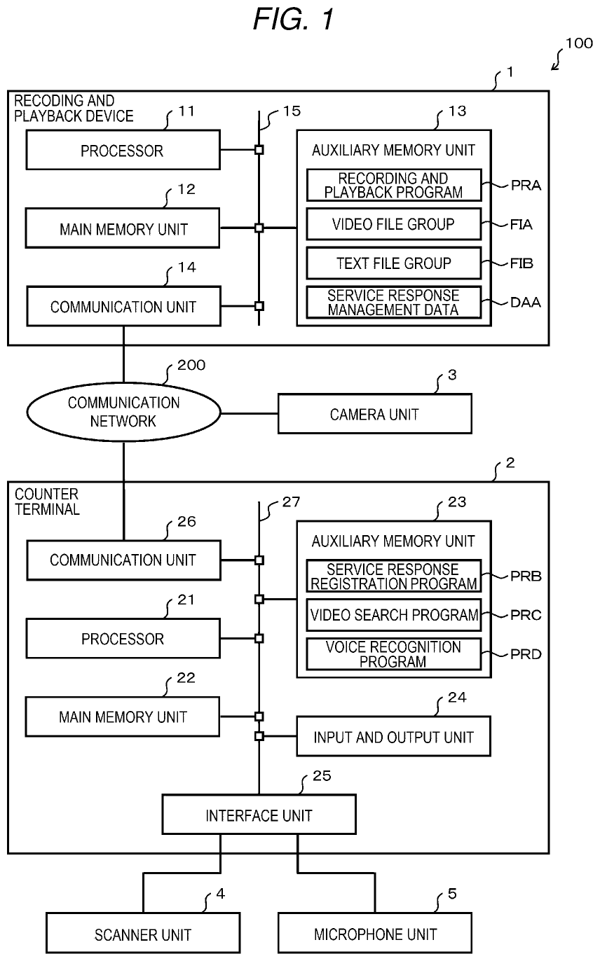

FIG. 1 is a block diagram showing a schematic configuration of a recording system 100 according to this embodiment, and a main circuit configuration of a recording and playback device 1 and a counter terminal 2 included in the recording system 100.

The recording system 100 includes the recording and playback device 1, the counter terminal 2, a camera unit 3, a scanner unit 4, and a microphone unit 5. The recording and playback device 1, the counter terminal 2, and the camera unit 3 are configured to be able to communicate with each other via a communication network 200. The scanner unit 4 and the microphone unit 5 are coupled to the counter terminal 2.

As the communication network 200, one or a suitable combination of the internet, a VPN (virtual private network), a LAN, a public communication network, a mobile communication network, and various other networks for computer communication can be used. In an example, the internet is used as the communication network 200.

The recording and playback device 1 is an information processing device that performs information processing to save and play back a video file and a text file in which a service response to patient by a store clerk at a counter where the counter terminal 2 is installed is recorded.

The counter terminal 2 is a terminal device installed in a pharmacy where a counter to provide a service response to patient to be recorded is provided. The counter terminal 2 performs various information processing, described later, in connection with a service response to patient by a store clerk.

The camera unit 3 captures an image over an image capture area defined in such a way that the state of a service response to patient by a store clerk at the counter is shown, and also takes in a voice around the image capture area, and thus acquires video data with voice. The camera unit 3 transmits the video data to the recording and playback device 1 via the communication network 200. As the camera unit 3, for example, an existing unit referred to a so-called network camera can be used. That is, the camera unit 3 has a function as an image capture unit.

The scanner unit 4 has a barcode scanner and outputs barcode data acquired by reading a barcode with this barcode scanner.

The microphone unit 5 has a patient-side microphone and a store clerk-side microphone. The patient-side microphone is provided with a directionality toward the position of the patient at the counter. The patient-side microphone receives an ambient sound mainly made up of the patient's voice and outputs voice data (hereinafter referred to as patient voice data). The store clerk-side microphone is provided with a directionality toward the position of the store clerk at the counter. The store clerk-side microphone receives an ambient sound mainly made up of the store clerk's voice and outputs voice data (hereinafter referred to as store clerk voice data). That is, the microphone unit 5 is an example of a voice collection unit that collects a voice around the counter, which is the image capture area of the camera unit 3.

Thus, the counter terminal 2, the camera unit 3, the scanner unit 4, and the microphone unit 5 form a device set to record a service response to a customer at one counter. While only one device set is shown in FIG. 1, a plurality of similar device sets may be provided at a plurality of corresponding counters, respectively.

The recording and playback device 1 has a processor 11, a main memory unit 12, an auxiliary memory unit 13, a communication unit 14, and a transmission path 15.

The processor 11, the main memory unit 12, and the auxiliary memory unit 13 are coupled together via the transmission path 15 and thus form a computer that executes information processing to implement functions as the recording and playback device 1.

The processor 11 is equivalent to a central unit of the computer. The processor 11 executes information processing to control each part in order to implement various functions as the recording and playback device 1 according to an information processing program such as an operating system or an application program.

The main memory unit 12 is equivalent to a main memory part of the computer. The main memory unit 12 includes a read-only memory area and a rewritable memory area. The main memory unit 12 stores a part of the foregoing information processing program in the read-only memory area. In some cases, the main memory unit 12 may store necessary data for the processor 11 to execute processing to control each part, in the read-only memory area or the rewritable memory area. In the main memory unit 12, the rewritable memory area is used as a work area of the processor 11.

The auxiliary memory unit 13 is equivalent to an auxiliary memory part of the computer. For example, an EEPROM (electrically erasable programmable read-only memory), an HDD (hard disk drive), an SSD (solid-state drive), or various other known memory devices can be used as the auxiliary memory unit 13. The auxiliary memory unit 13 stores data used by the processor 11 to perform various processing and data generated as a result of processing by the processor 11. In some cases, the auxiliary memory unit 13 may store the foregoing information processing program. In this embodiment, the auxiliary memory unit 13 stores a recording and playback program RPA, which is an information processing program. The recording and playback program RPA is an application program that describes procedures of information processing, described later, to record a video and text and to play back a recorded video and text (hereinafter referred to recording and playback processing). A part of the memory area of the auxiliary memory unit 13 is used as an area for storing a video file group FIA, a text file group FIB, and service response management data DAA. The video file group FIA is a data file group formed by aggregating video files formed of video data transmitted from the camera unit 3 and converted in data files in a predetermined file format. The text file group FIB is a data file group formed by aggregating text files transmitted from the counter terminal 2 as will be described later. The service response management data DAA is data for managing a service response to patient provided at the counter. Details of the service response management data will be described later.

The communication unit 14 executes communication processing to perform data communication via the communication network 200. As the communication unit 14, for example, an existing communication device for the internet can be used.

The transmission path 15 includes an address bus, a data bus, and a control signal line or the like and transmits data and control signals sent and received between the components coupled thereto.

As the hardware of the recording and playback device 1, for example, a computer device for a general-purpose server device can be used. In this case, the recording and playback device 1 is generally provided in the state where the recording and playback program RPA is stored in the auxiliary memory unit 13. However, the hardware of the recording and playback device 1 in the state where the recording and playback program RPA is not stored in the auxiliary memory unit 13 and the recording and playback program RPA may be separately provided. Then, the recording and playback program RPA may be written into the auxiliary memory unit 13 in response to an operation by any worker. Alternatively, the hardware of the recording and playback device 1 in the state where an information processing program of the same type as the recording and playback program RPA but a different version is stored in the auxiliary memory unit 13 and the recording and playback program RPA may be separately provided. Then, the recording and playback program RPA may be written in the form of replacing the information processing program already stored in the auxiliary memory unit 13. The recording and playback program RPA can be provided in the form of being recorded in a removable recording medium such as a magnetic disk, a magneto-optical disk, an optical disk or a semiconductor memory, or can be provided by communication via a network. The recording and playback program RPA may be stored in the main memory unit 12.

The counter terminal 2 has a processor 21, a main memory unit 22, an auxiliary memory unit 23, an input and output unit 24, an interface unit 25, a communication unit 26, and a transmission path 27.

The functions of the processor 21, the main memory unit 22, the auxiliary memory unit 23, the communication unit 26, and the transmission path 27 are substantially equivalent to the functions of the processor 11, the main memory unit 12, the auxiliary memory unit 13, the communication unit 14, and the transmission path 15. However, the auxiliary memory unit 23 does not store the recording and playback program RPA but stores a service response registration program PRB, a video search program PRC, and a voice recognition program PRD. The service response registration program PRB is an application program that describes procedures of information processing, described later, to register a service response to patient to manage the execution status of the service response to patient (hereinafter referred to as service response registration processing). The video search program PRC is an application program that describes procedures of information processing for a user interface used to search a video recorded by the recording and playback device 1 (hereinafter referred to as search processing). The voice recognition program PRD is an application program that describes procedures of information processing, described later, to control voice recognition processing on voice data outputted from the microphone unit 5 (hereinafter referred to as voice recognition processing).

The input and output unit 24 has at least one of various user interface devices. The input and output unit 24 has, for example, a touch panel and a sound device as the user interface device. The input and output unit 24 may have various devices, for example, a mouse, a keyboard or the like, as the user interface device.

The interface unit 25 has a port which an external device is attachable to and removable from. The scanner unit 4 and the microphone unit 5 are coupled to this port. The interface unit 25 provides an interface to transmit and receive data to and from a device installed at the port, such as the scanner unit 4 and the microphone unit 5. As the interface unit 25, for example, an existing device conforming to an existing standard such as the USB (universal serial bus) standard can be used.

As the hardware of the counter terminal 2, various types of information processing terminals such as a desktop computer, a book-type computer, a tablet computer or a smartphone can be used. As the counter terminal 2, generally, hardware in the state where the service response registration program PRB, the video search program PRC, and the voice recognition program PRD are not stored in the auxiliary memory unit 23, and each program, are separately provided. Each program is then written into the auxiliary memory unit 23 in response to an operation by any worker. However, the counter terminal 2 may be provided in the state where the service response registration program PRB, the video search program PRC, and the voice recognition program PRD are stored in the auxiliary memory unit 23. Alternatively, the hardware of the counter terminal 2 in the state where information processing programs of the same type as the service response registration program PRB, the video search program PRC, and the voice recognition program PRD but a different version are stored in the auxiliary memory unit 23, and the service response registration program PRB, the video search program PRC, and the voice recognition program PRD, may be separately provided. Then, the service response registration program PRB, the video search program PRC, and the voice recognition program PRD may be written in the form of replacing the information processing programs already stored in the auxiliary memory unit 23. The service response registration program PRB, the video search program PRC, and the voice recognition program PRD can be provided in the form of being recorded in a removable recording medium such as a magnetic disk, a magneto-optical disk, an optical disk or a semiconductor memory, or can be provided by communication via a network. The service response registration program PRB, the video search program PRC, and the voice recognition program PRD may be stored in the main memory unit 22.

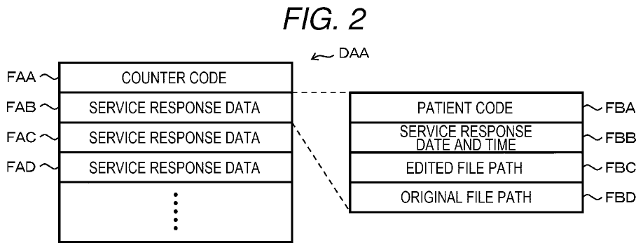

FIG. 2 schematically shows the data structure of the service response management data DAA.

If the recording and playback device 1 records a service response to patient at a plurality of counters, a plurality of service response management data DAA correlated with the plurality of counters, respectively, are stored in the auxiliary memory unit 13.

The service response management data DAA includes fields FAA, FAB, FAC, and so on. In the field FAA, a counter code as an identifier of a correlated counter is set. In each of the fields from FAB onward, service response data can be set. How many pieces of service response data are included in the service response management data DAA varies according to the operation status of the recording and playback device 1. The service response data is correlated with a service response to patient on one occasion and is an aggregate of various data about this service response to patient.

As shown in FIG. 2, one piece of service response data includes fields FBA, FBB, FBC, FBD. In the field FBA, a patient code as an identifier of a patient who is a target of a correlated service response to patient is set. In the field FBB, a start date and time of the correlated service response to patient is set. In the field FBC, a file path to specify an edited text file, described later, about the correlated service response to patient (hereinafter referred to as an edited file path) is set. In the field FBD, a file path to specify an original text file, described later, about the correlated service response to patient (hereinafter referred to as an original file path) is set.

Operations of the recording system 100 configured as described above will now be described. The contents of various processing described below are an example. A change in the order of a part of the processing, an omission of a part of the processing, and an addition of another processing or the like can be made according to need.

At the counter terminal 2, if an instruction to execute the service response registration processing is given by a predetermined operation of the input and output unit 24, the processor 21 starts the service response registration processing, based on the service response registration program PRB.

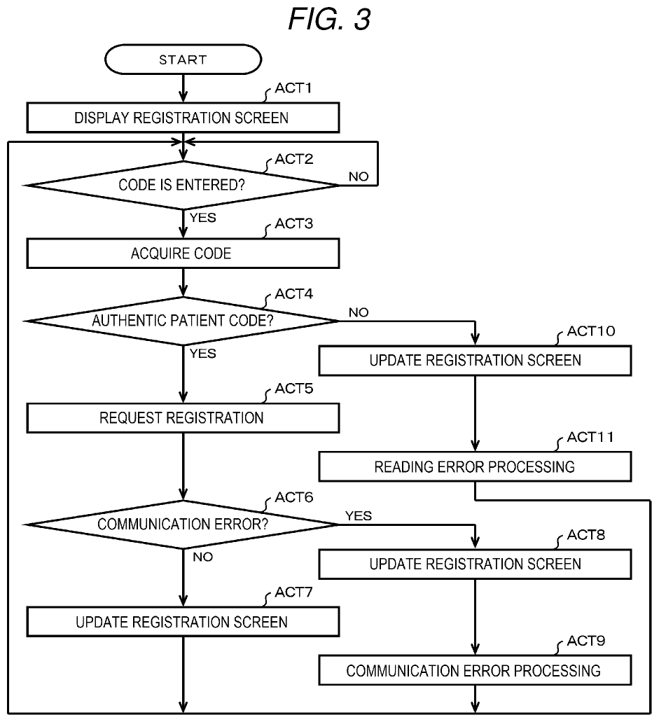

FIG. 3 is a flowchart of the service response registration processing.

In ACT 1, the processor 21 displays a registration screen on the touch panel provided in the input and output unit 24. The registration screen is a GUI (graphical user interface) screen for a store clerk to start and register a service response to patient.

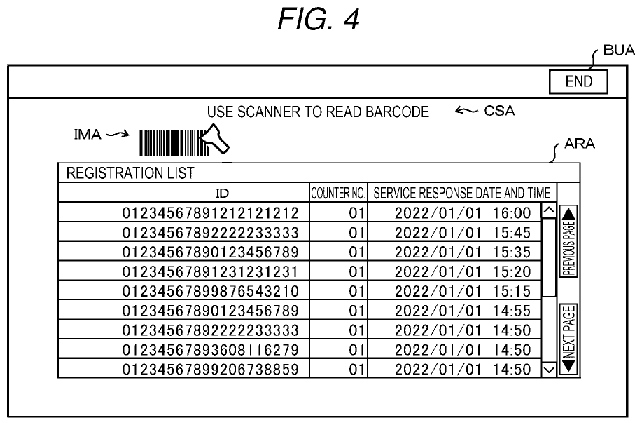

FIG. 4 shows an example of the registration screen.

In various screens shown in FIG. 4 and other drawings, the illustration of a part of display objects may be omitted.

As shown in FIG. 4, the registration screen includes a character string CSA, an image IMA, a button BUA, and an area ARA.

The character string CSA shows a text message that guides the store clerk to read a barcode shown in a prescription brought in by a patient who is a service response target. The image IMA is an image to cause the store clerk to imagine a task that the store clerk should carry out. The button BUA is a soft key for the operator to give an instruction to end the service response registration processing. In the area ARA, a list of service responses to patient registered up to this point is shown. When the processor 21 executes ACT 1, no service response to patient is registered yet and therefore no specific information should be shown in the list shown in the area ARA. However, in FIG. 4, a state where specific information is shown is illustrated in order to clarify how the list in the area ARA looks. The list shown in the area ARA includes a patient code as an identifier of a patient who is a service response target, a counter code, and a service response date and time, in connection with each service response to patient.

To generate the registration screen, the processor 21 may extract information to be shown in the list of service responses to patient from service response history data stored in the auxiliary memory unit 23 or from service response history data managed by another information processing device. As this another information processing device, a POS (point-of-sale) server that performs management processing of sales of medicines at a pharmacy can be used.

To start a service response to patient for a new patient, the store clerk in charge of the service response to patient uses a barcode scanner provided in the scanner unit 4 to read a barcode shown in a prescription brought in by the patient. After scanning the barcode with the barcode scanner and acquiring barcode data, the scanner unit 4 outputs the barcode data.

After displaying the registration screen in ACT 1 in FIG. 3, the processor 21 proceeds to ACT 2.

In ACT 2, the processor 21 waits for a code to be entered. If barcode data outputted from the scanner unit 4 as described above is taken in by the interface unit 25, the processor 21 gives YES, determining that a code is entered, and then proceeds to ACT 3.

In ACT 3, the processor 21 attempts to acquire a patient code included in the barcode data. That is, since a patient code of a patient who is a target to which a medicine is prescribed by a prescription is included at a predetermined position in barcode data represented by a barcode shown in the prescription, the processor 21 extracts the data at the foregoing position as the patient code.

In ACT 4, the processor 21 checks whether an authentic patient code is acquired or not. If the authentic patient code is acquired, the processor 21 gives YES and proceeds to ACT 5.

In ACT 5, the processor 21 requests the recording and playback device 1 to register a new service response to patient. For example, the processor 21 sends out predetermined request data for the request addressed to the recording and playback device 1, from the communication unit 26 to the communication network 200. The processor 21 includes, for example, the patient code, the counter code as an identifier of the counter where the counter terminal 2 is used, and the service response date and time, into the request data. Which date and time to be employed as the service response date and time may be suitably decided, for example, by the generator of the service response registration program PRB or the like. However, it is assumed that the date and time related to the timing when YES is given in ACT 2 or the timing of execution of one of ACTS 3 to 5 is employed.

The scan of the barcode in the prescription for the transaction registration to the POS server performing the management processing of the sales of medicines and the scan of the barcode by the store clerk may be a common process. In this case, the processor 21 in ACT 5 also requests the POS server to perform the transaction registration.

As the foregoing request data is transmitted to the recording and playback device 1 via the communication network 200, the communication unit 14 receives the request data.

At the recording and playback device 1, basically, the processor 11 constantly executes the recording and playback processing based on the recording and playback program RPA, except during a particular period such as during maintenance work. However, the processor 11 may execute the recording and playback processing during a predetermined service providing time period only.

If the recording system 100 has a plurality of device sets, each being formed of the counter terminal 2, the camera unit 3, the scanner unit 4, and the microphone unit 5, at a plurality of corresponding counters, respectively, the recording and playback processing corresponding to the plurality of counters, respectively, are executed in parallel. However, in the description below, only the recording and playback processing corresponding to one counter provided with one device set shown in FIG. 1 will be described.

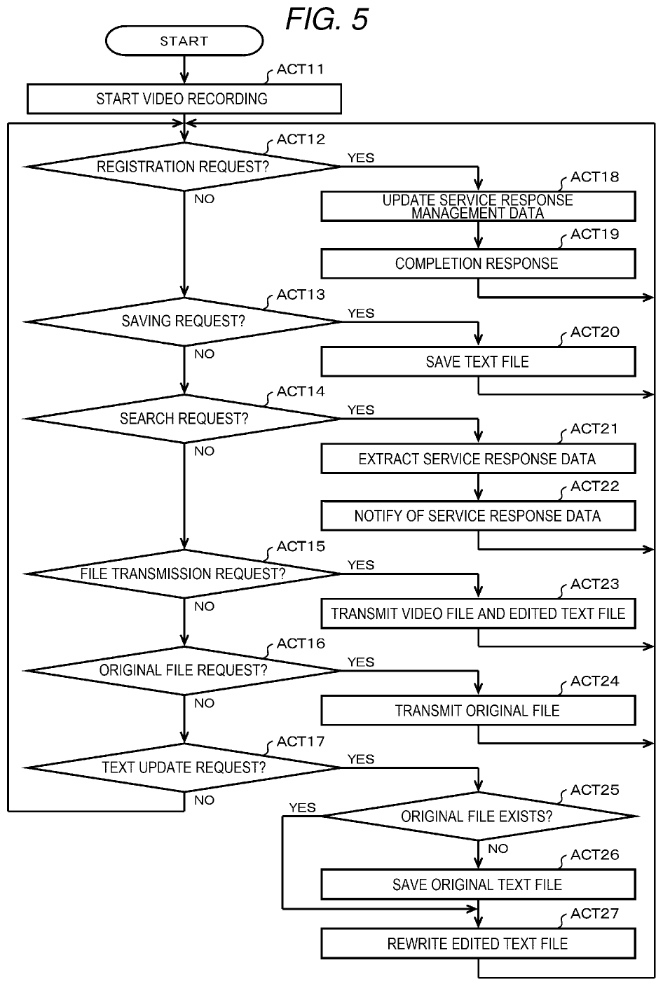

FIG. 5 is a flowchart of the recording and playback processing.

In ACT 11, the processor 11 starts video recording. That is, the processor 11 converts video data with voice transmitted from the camera unit 3, transmitted via the communication network 200, and received by the communication unit 14, into a video file of a predetermined multimedia format, and adds the video file to the video file group FIA. In an example, the processor 11 extracts video data every five minutes and converts the extracted video data into one video file of the mp4 format. The processor 11 includes, into each video file, time information representing the start date and time and the end date and time of the video represented the video file.

In this way, the processor 11 acquires video data transmitted from the camera unit 3 and stores a video file including the video data, in the auxiliary memory unit 13. That is, as the processor 11 executes the information processing based on the recording and playback program RPA, the computer whose central unit is the processor 11 functions as a video acquisition unit. The auxiliary memory unit 13 storing the video file group FIA including the video file is an example of a video memory unit.

The processor 11 shifts to the waiting states of ACT 12, ACT 13, ACT 14, ACT 15, ACT 16, ACT 17 while performing the video recording started as described above. In the waiting states of ACTS 12 to 17, the processor 11 waits for one of a registration request, a saving request, a search request, a file transmission request, an original file request, and a text update request to be made.

If the request data transmitted from the counter terminal 2 to request registration as described above is received by the communication unit 14, the processor 11 gives YES in ACT 12, determining that registration is requested, and then proceeds to ACT 18.

In ACT 18, the processor 11 updates the service response management data DAA. That is, the processor 11 sets, for example, the patient code and the service response date and time included in the request data, in the fields FBA, FBB. The processor 11 leaves the fields FBC, FBD blank, in the new service response data generated at this point, or sets a predetermined null value in these fields. The processor 11 may generate service response data made up of the fields FBA, FBB only.

In ACT 19, the processor 11 gives a response to the effect that the registration corresponding to the registration request is complete, to the counter terminal 2. The processor 11 sends out, for example, predetermined response data for a completion response addressed to the counter terminal 2, from the communication unit 14 to the communication network 200. As the response data is transmitted to the counter terminal 2 via the communication network 200, the communication unit 26 receives this response data. After that, the processor 11 returns to the waiting states of ACTS 12 to 17.

At the counter terminal 2, after requesting registration in ACT 5 in FIG. 3, the processor 21 proceeds to ACT 6.

In ACT 6, the processor 21 checks whether a communication error has occurred in the foregoing registration request or not. If the response data for the completion response is received by the communication unit 26 as described above, the processor 21 gives NO, determining that there is no communication error, and then proceeds to ACT 7.

In ACT 7, the processor 21 updates the registration screen to reflect the result of the registration of the service response of this time. After that, the processor 21 returns to the waiting state of ACT 2.

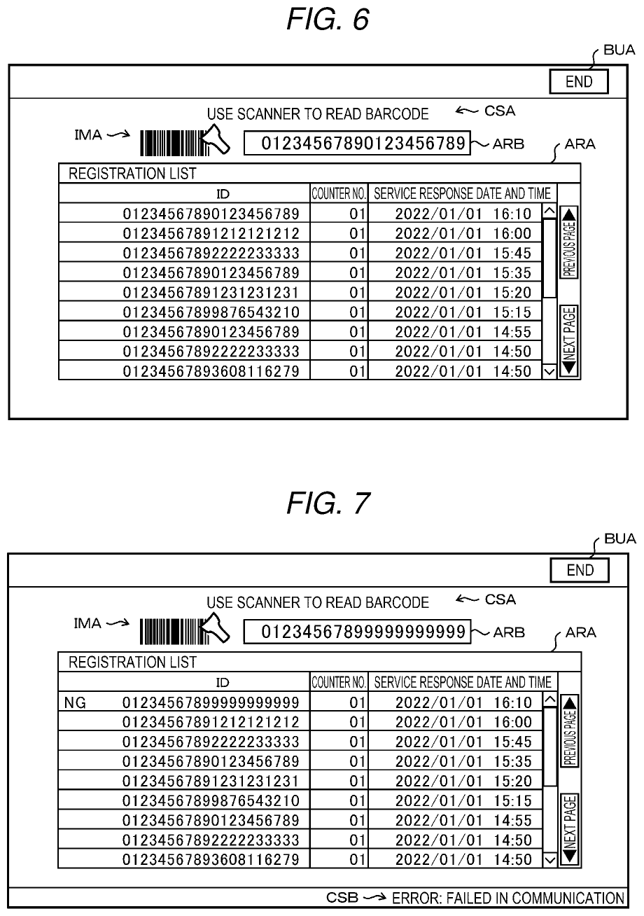

FIG. 6 shows an example of the registration screen.

FIG. 6 is an example where a service response to a patient with a patient code “01234567890123456789” is started from the state where the registration screen shown in FIG. 4 is displayed.

On the registration screen shown in FIG. 6, an area ARB is added to the registration screen shown in FIG. 4 and the patient code acquired in ACT 3 is shown in this area ARB. On the registration screen shown in FIG. 6, the result of the registration of this time is shown in the first line of the list shown in the area ARA.

Meanwhile, if it is confirmed that a communication error of some kind has occurred, the processor 21 gives YES in ACT 6 and proceeds to ACT 8.

In ACT 8, the processor 21 updates the registration screen to show that a communication error has occurred.

FIG. 7 shows an example of the registration screen.

FIG. 7 is an example where a communication error has occurred in the registration request for a service response to a patient with a patient code “01234567899999999999” from the state where the registration screen shown in FIG. 4 is displayed.

On the registration screen shown in FIG. 7, an area ARB is added to the registration screen shown in FIG. 4 and the patient code acquired in ACT 3 is shown in this area ARB. On the registration screen shown in FIG. 7, the result of the registration of this time is shown in the first line of the list shown in the area ARA and includes a character string “NG” in order to indicate that an error has occurred. On the registration screen shown in FIG. 7, a character string CSB is added to the registration screen shown in FIG. 4. The character string CSB shows a text message that a communication error has occurred.

After updating the registration screen in ACT 8 in FIG. 3, the processor 21 proceeds to ACT 9.

In ACT 9, the processor 21 performs communication error processing. As the communication error processing, for example, the processor 21 performs processing to cause the sound device included in the input and output unit 24 to operate and output a voice message such as “communication error”. Alternatively, as the communication error processing, for example, the processor 21 may perform another suitable processing such as processing to cause a warning lamp included in the input and output unit 24 to flash on and off. After that, the processor 21 returns to the waiting state of ACT 2.

If an inappropriate barcode, such as a barcode shown in a prescription that is not a target of a service response at the counter where the counter terminal 2 is used, is scanned, the code acquired in ACT 3 is not an authentic patient code. Therefore, in this case, the processor 21 gives NO in ACT 4 in FIG. 3 and proceeds to ACT 10.

In ACT 10, the processor 21 updates the registration screen to show that a reading error has occurred.

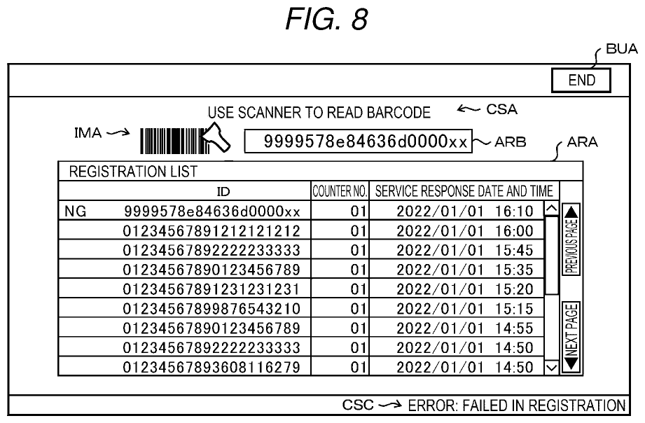

FIG. 8 shows an example of the registration screen.

FIG. 8 is an example where a code “9999578e84636d0000xx”, which is of a different system from an authentic patient code, is acquired from the state where the registration screen shown in FIG. 4 is displayed.

On the registration screen shown in FIG. 8, an area ARB is added to the registration screen shown in FIG. 4 and the patient code acquired in ACT 3 is shown in this area ARB. On the registration screen shown in FIG. 8, the result of the registration of this time is shown in the first line of the list shown in the area ARA and includes a character string “NG” in order to indicate that an error has occurred. On the registration screen shown in FIG. 8, a character string CSC is added to the registration screen shown in FIG. 4. The character string CSC shows a text message that the registration has failed.

After updating the registration screen in ACT 10 in FIG. 3, the processor 21 proceeds to ACT 11.

In ACT 11, the processor 21 performs reading error processing. As the reading error processing, for example, the processor 21 performs processing to cause the sound device included in the input and output unit 24 to operate and output a voice message such as “failed in registration”. Alternatively, as the reading error processing, for example, the processor 21 may perform another suitable processing such as processing to cause the warning lamp included in the input and output unit 24 to flash on and off. After that, the processor 21 returns to the waiting state of ACT 2.

If the registration 4 the service response is successful, the store clerk subsequently carries out the service response to patient based on the prescription. This service response to patient is carried out, for example, according to the following procedures. That is, the store clerk first requests a medicine preparation staff to prepare the medicine based on the prescription. Next, when the preparation of the medicine is complete, the store clerk hands over the prepared medicine to the patient while giving an explanation. The store clerk then settles the payment for the medicine.

At the counter terminal 2, if an instruction to execute the voice recognition processing is given by a predetermined operation of the input and output unit 24, the processor 21 starts the voice recognition processing based on the voice recognition program PRD. The store clerk may cause the voice recognition processing to be constantly executed during a period when the service response to patient can be provided, or may start the execution of the voice recognition processing according to need.

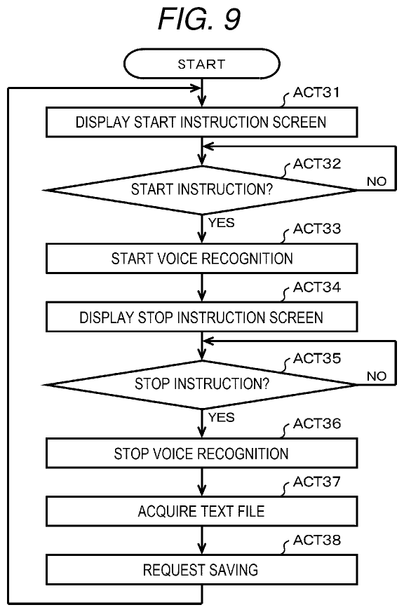

FIG. 9 is a flowchart of the voice recognition processing.

In ACT 31, the processor 21 displays a start instruction screen on the touch panel provided in the input and output unit 24. The start instruction screen is a GUI screen to accept an instruction to start voice recognition.

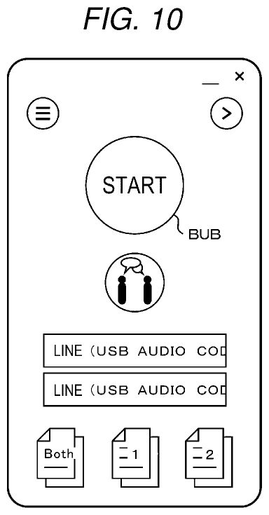

FIG. 10 shows an example of the start instruction screen.

The start instruction screen includes a plurality of display objects as shown in FIG. 10. One of the display objects is a button BUB. The button BUB is a soft key for the operator to give an instruction to start voice recognition.

If the store clerk wants to start the voice recognition of a voice corresponding to the service response to patient, the store clerk gives an instruction to start the voice recognition, for example, by a predetermined operation such as a tap on the button BUB.

After displaying the start instruction screen in ACT 31 in FIG. 9, the processor 21 proceeds to ACT 32.

In ACT 32, the processor 21 waits for a start instruction. If an instruction to start the voice recognition is given as described above, the processor 21 gives YES and proceeds to ACT 33.

In ACT 33, the processor 21 starts the voice recognition. For example, the processor 21 requests a cloud server, not shown in FIG. 1, to start the voice recognition. After that, the processor 21 transmits the patient voice data and the store clerk voice data outputted from the microphone unit 5, to the cloud server. The processor 21 may execute information processing to generate text data representing the content of an utterance included in the voice represented by each voice data in connection with the patient voice data and the store clerk voice data, according to the voice recognition program PRD or another information processing program.

In ACT 34, the processor 21 displays a stop instruction screen on the touch panel provided in the input and output unit 24. The stop instruction screen is a GUI screen to accept an instruction to stop voice recognition.

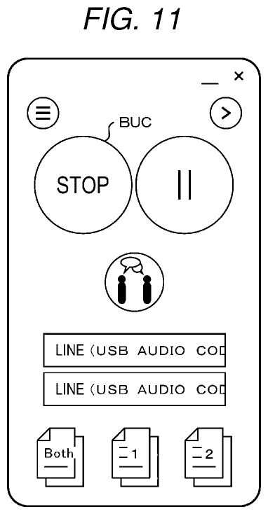

FIG. 11 shows an example of the stop instruction screen.

The stop instruction screen includes a plurality of display objects as shown in FIG. 11. One of the display objects is a button BUC. The button BUC is a soft key for the operator to give an instruction to stop voice recognition.

If the store clerk wants to stop the voice recognition of a voice corresponding to the service response to patient, the store clerk gives an instruction to stop the voice recognition, for example, by a predetermined operation such as a tap on the button BUC.

After displaying the stop instruction screen in ACT 34 in FIG. 9, the processor 21 proceeds to ACT 35.

In ACT 35, the processor 21 waits for a stop instruction. For example, while waiting for a stop instruction in this way, the processor 21 continues transmitting the patient voice data and the store clerk voice data outputted from the microphone unit 5, to the cloud server. If an instruction to stop the voice recognition is given as described above, the processor 21 gives YES and proceeds to ACT 36.

In ACT 36, the processor 21 stops the voice recognition. That is, for example, the processor 21 ends the transmission of the patient voice data and the store clerk voice data to the foregoing cloud server and then requests the cloud server to stop the voice recognition.

In ACT 37, the processor 21 acquires a text file generated based on the voice recognition from the foregoing cloud server. If the cloud server transmits, in real time, text data generated based on the voice recognition, the processor 21 may collect the text data during the waiting state of ACT 35 and convert the collected text data into a text file in ACT 37.

The text file shows, for example, contents such as “2022/01/01 16:10:10, 1, Can I help you?, 2022/01/01 16:10:13, 2, Please take care of this, 2022/01/01 16/10/20, 1, I'll take your prescription, 2022/01/01 16/10/31, 2, Ah medication record book, 2022/01/01 16/10/37, 1, Thank you, 2022/01/01 16/10/40, 1, I'll take it, 2022/01/01 16/10/45, 1, Please have a seat and wait”. In this case, for example, “2022/01/01 16:10:10” of the text “2022/01/01 16:10:10, 1, Can I help you?” indicates the date and time of the voice of the recognition target. “1” indicates that this is the result of recognition from the store clerk voice data. “Can I help you?” represents text data that is the result of recognition. “2” of the text “2, Please take care of this” indicates that this is the result of recognition from the patient voice data.

In ACT 38, the processor 21 requests the recording and playback device 1 to save the text file. For example, the processor 21 includes the text file acquired in ACT 37 and data representing the period when the voice data that is the target of voice recognition is acquired (hereinafter referred to as recognition period data), into request data for this request. After that, the processor 21 repeats the processing from ACT 31 onward, similarly to the above.

At the recording and playback device 1, if the request data for the request to save the text file is received by the communication unit 14, the processor 11 gives YES in ACT 13 in FIG. 5, determining that a saving request is made, and then proceeds to ACT 20.

In ACT 20, the processor 11 saves the text file in response to the request from the foregoing counter terminal 2. That is, for example, the processor 11 adds the text file included in the request data, as an edited text file, to the text file group FIB in correlation with the recognition period data similarly included in the request data. The edited text file is a text file that can be updated by editing processing, described later. The processor 11 sets the file path of the foregoing saved edited text file, as an edited file path, in the field FBC of the service response data set in the last field of the service response management data DAA. After that, the processor 11 returns to the waiting states of ACTS 12 to 17.

The processor 11 thus acquires the text file, which is an example of text data, and stores this text file in the auxiliary memory unit 13. That is, as the processor 11 executes the information processing based on the recording and playback program RPA, the computer whose central unit is the processor 11 functions as a text acquisition unit. The auxiliary memory unit 13 storing the text file group FIB including the text file, which is an example of text data, is an example of a text memory unit.

In this way, the state of the service response to patient at the counter is recorded as a video file with a voice file and as a text file representing the result of voice recognition, and the status of implementation of the service response to patient is recorded as the service response management data DAA.

At the counter terminal 2, if an instruction to execute the search processing is given by a predetermined operation to the input and output unit 24 by a checker who attempts to check a past service response to patient carried out by the store clerk or the manager or the like, the processor 21 starts the search processing based on the video search program PRC.

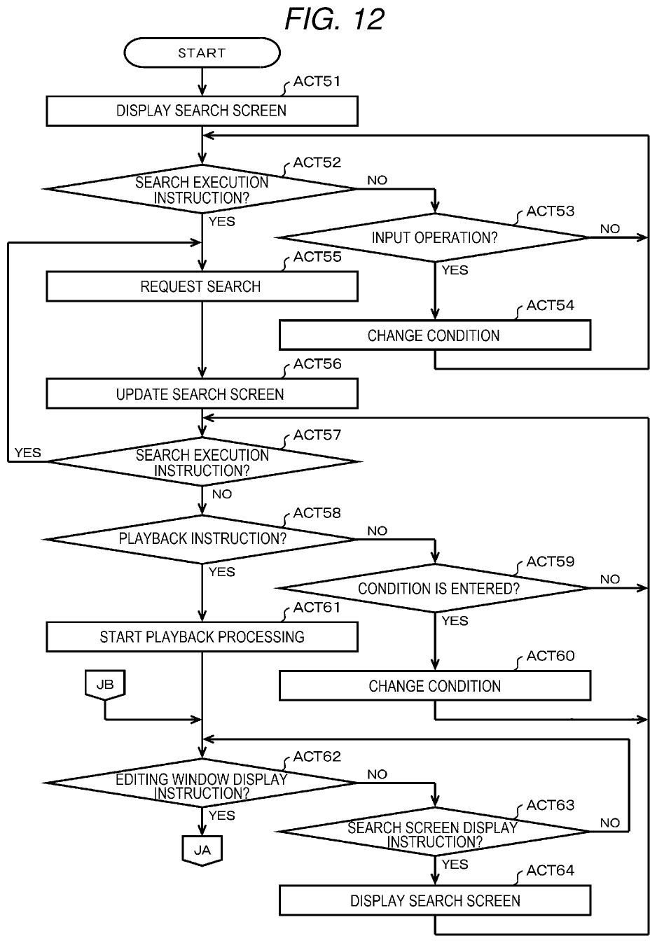

FIGS. 12, 13, and 14 are flowcharts of the search processing.

In ACT 51 in FIG. 12, the processor 21 displays a search screen on the touch panel provided in the input and output unit 24. The search screen is a GUI screen for searching a service response to patient.

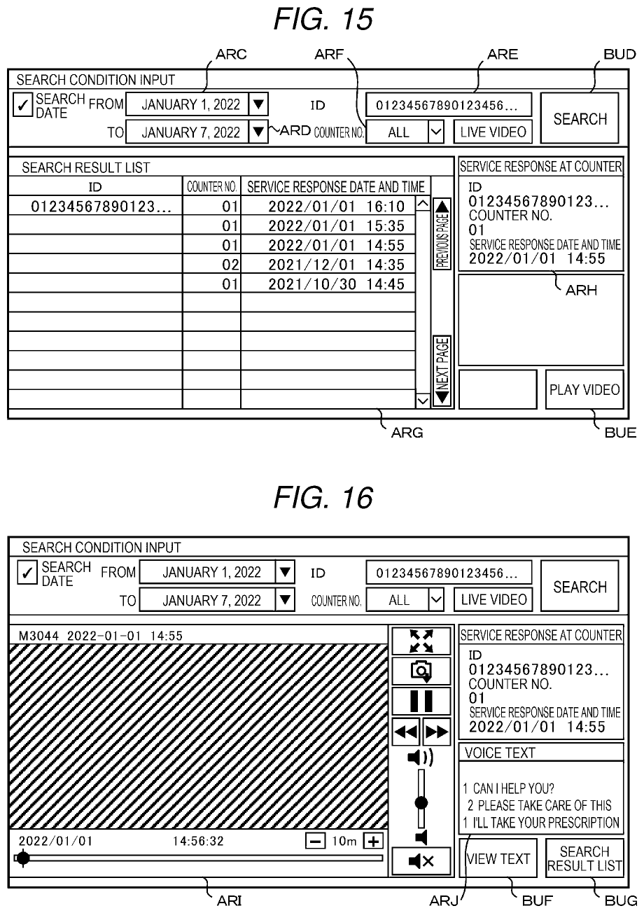

FIG. 15 shows an example of the search screen.

The search screen shown in FIG. 15 includes areas ARC, ARD, ARE, ARF, ARG, ARH and buttons BUD, BUE. The areas ARC, ARD, ARE, ARF show search conditions that are already entered. The area ARG shows a list of service responses to patient that match the search conditions. The area ARH shows information about one selected service response to patient. The button BUD is a soft key for the operator to give an instruction to execute the search. The button BUE is a soft key for the operator to give an instruction to play the video.

When the processor 21 executes ACT 51, no search condition is designated yet and the search is not executed yet, either. Therefore, there is no information to be shown in the areas ARC, ARD, ARE, ARF, ARG, ARH and these areas are left blank. However, in FIG. 15, a state where specific information is shown is illustrated in order to clarify how various information is shown in the areas ARC, ARD, ARE, ARF, ARG, ARH.

After displaying the search screen in ACT 51 in FIG. 12, the processor 21 proceeds to the waiting states of ACT 52 and ACT 53.

In ACT 52 and ACT 53, the processor 21 waits for an instruction to execute search or an input operation to enter a search condition.

The store clerk performs a predetermined operation to enter a condition for narrowing down the service responses recorded by the recording and playback device 1 to some service responses, as a search condition, for example, with the input and output unit 24. In response to this operation, the processor 21 gives YES in ACT 53 and proceeds to ACT 54.

In ACT 54, the processor 21 changes the search condition in response to the performed operation. The processor 21 suitably updates the information shown in the areas ARC, ARD, ARE, ARF, ARG, ARH so as to show the search conditions after the change. After that, the processor 21 returns to the waiting states of ACT 52 and ACT 53.

After finishing the setting of desired search conditions, the checker gives an instruction to execute the search, for example, by a predetermined operation such as a tap on the button BUD in the search screen. In response to this instruction, the processor 21 gives YES in ACT 52 and proceeds to ACT 55.

In ACT 55, the processor 21 requests the recording and playback device 1 to execute the search. For example, the processor 21 includes data representing the set search conditions into request data for this request.

At the recording and playback device 1, if the request data for the search request is received by the communication unit 14, the processor 11 gives YES in ACT 14 in FIG. 5, determining that a search request is made, and then proceeds to ACT 21.

In ACT 21, the processor 11 extracts all service response data that match the search conditions represented by the data included in the request data, from the service response management data DAA.

In ACT 22, the processor 11 notifies the counter terminal 2 of the foregoing extracted service response data. That is, the processor 11 sends out notification data including the foregoing extracted service response data addressed to the counter terminal 2, from the communication unit 14 to the communication network 200. After that, the processor 11 returns to the waiting states of ACTS 12 to 17.

If the foregoing notification data is transmitted to the counter terminal 2 via the communication network 200, the communication unit 26 receives the notification data.

At the counter terminal 2, after requesting the search in ACT 55 in FIG. 12, the processor 21 proceeds to ACT 56.

In ACT 56, the processor 21 updates the search screen in such a way that a list of respective service responses to patient of the service response data included in the foregoing notification data is shown in the area ARG. On the search screen at this stage, no specific information about the service response to patient is shown in the area ARH.

In the state where the updated search screen is displayed, the processor 21 enters the waiting states of ACT 57, ACT 58, and ACT 59. In the waiting states of ACTS 57 to 59, the processor 21 waits for an instruction to execute the search, an instruction to play back, or an input operation to enter a search condition.

To change a search condition and search again, the checker performs a predetermined operation to enter a search condition. In response to this operation, the processor 21 gives YES in ACT 59 and proceeds to ACT 60.

In ACT 60, the processor 21 changes the search condition in response to the performed operation. The processor 21 suitably updates the information shown in the areas ARC, ARD, ARE, ARF, ARG, ARH so as to show the search conditions after the change. After that, the processor 21 returns to the waiting states of ACTS 57 to 59.

After finishing the change to set desired search conditions, the checker gives an instruction to execute the search, for example, by a predetermined operation such as a tap on the button BUD in the search screen. In response to this instruction, the processor 21 gives YES in ACT 57 and repeats the processing from ACT 55 onward, similarly to the above.

If the checker finds a service response to patient that should be checked, the checker designates the service response to patient by a predetermined operation and then gives an instruction to play the video, for example, by a predetermined operation such as a tap on the button BUE in the search screen. In response to this instruction, the processor 21 gives YES in ACT 58 and proceeds to ACT 61.

In ACT 61, the processor 21 starts playback processing to play the video corresponding to the designated service response to patient. That is, for example, the processor 21 acquires a video file corresponding to a period including the service response date and time of the designated service response to patient, from the recording and playback device 1. The processor 21 also acquires an edited text file correlated with recognition period data representing a recognition period including the start date and time of the acquired video file, from the recording and playback device 1. Specifically, for example, the processor 21 requests the recording and playback device 1 to send a file, with a notification of the service response date and time of the designated service response to patient. The processor 21 then displays, on the touch panel provided in the input and output unit 24, a playback screen showing a video and a text represented by the video file and the edited text file, respectively, sent from the recording and playback device 1 in response to this request, while synchronizing a frame and a text corresponding to the same date and time. The processor 21 also outputs a voice based on the voice data included in the video file, from the sound device provided in the input and output unit 24.

At the recording and playback device 1, if the processor 11 receives the request to send a file as described above, the processor 11 gives YES in ACT 15 in FIG. 5 and proceeds to ACT 23.

In ACT 23, the processor 11 extracts a video file and an edited text file corresponding to the request, from the video file group FIA and the text file group FIB, and transmits the video file and the edited text file to the counter terminal 2. For example, the processor 11 extracts a video file in which the start date and time and the end date and time represented by the time information included in the video file are before and after the service response date and time which the processor 11 is notified of by the request, as a transmission target from the video file group FIA. The processor 11 may also extract, as a transmission target, another predetermined video file corresponding to timings before and after the foregoing video file that is a transmission target. That is, for example, the processor 11 may extract a video file in which the start date and time and the end date and time represented by the time information included in the video file are before and after the service response date and time which the processor 11 is notified of by the request, and one video file corresponding to a timing immediately before the above video file and one video file corresponding to a timing immediately after the above video file, as transmission targets. The processor 11 also finds out service response data in which the service response date and time which the processor 11 is notified of by the request is set in the field FBB, from among the service response data included in the service response management data DAA in which the counter code correlated with the counter terminal 2 is set in the field FAA. The processor 11 then extracts an edited text file specified by the edited file path set in the field FBC of this service response data, as a transmission target from the text file group FIB. The processor 11 sends out the video file and the edited text file thus extracted as transmission targets, addressed to the counter terminal 2, from the communication unit 14 to the communication network 200. After that, the processor 11 returns to the waiting states of ACTS 12 to 17.

FIG. 16 shows an example of the playback screen.

The playback screen shown in FIG. 16 is an example of a screen displayed in response to a tap on the button BUE in the search screen in the state shown in FIG. 15.

The playback screen includes areas ARI, ARJ and buttons BUF, BUG. The playback screen includes display objects equivalent to the areas ARC, ARD, ARE, ARF, ARG, ARH and the button BUD in the search screen shown in FIG. 15. However, these display objects are, for example, grayed out or the like and thus not to be operated.

The area ARI displays the currently played video in a hatched area and shows a plurality of GUI objects to control video playback. The area ARJ shows the text represented by the edited text file. The button BUF is a soft key for the operator to give an instruction to display an editing window. The button BUG is a soft key for the operator to give an instruction to display the search screen.

By thus displaying the playback screen, the processor 21 displays the video represented by the video file, which is video data. Thus, as the processor 21 executes the information processing based on the video search program PRC, the computer whose central unit is the processor 21 functions as a video display unit. Also, by displaying the playback screen, the processor 21 displays the text representing the voice collected at the time of capturing the displayed video. Thus, as the processor 21 executes the information processing based on the video search program PRC, the computer whose central unit is the processor 21 functions as a text display unit.

The checker can check the contents of the utterances by the store clerk and the patient in the service response to patient, based on the voice outputted from the sound device and the text displayed in the area ARJ.

However, there is a risk that the text displayed in the area ARJ may differ from the actual contents of the utterances due to an error in the voice recognition. If the checker notices such an error and thinks that the error should be corrected, the checker gives an instruction to display the editing window, for example, by a predetermined operation such as a tap on the button BUF. If the checker wants to check the search screen again, the checker gives an instruction to display the search screen, for example, by a predetermined operation such as a tap on the button BUG.

After starting the playback processing in ACT 61 in FIG. 12, the processor 21 enters the waiting states of ACT 62 and ACT 63 while continuing this playback processing. In the waiting states of ACT 62 and ACT 63, the processor 21 waits for an instruction to display the editing window or the search screen. If an instruction to display the search screen is given as described above, the processor 21 gives YES in ACT 63 and proceeds to ACT 64.

In ACT 64, the processor 21 displays the search screen displayed before the display of the playback screen in ACT 61, again on the touch panel. After that, the processor 21 returns to the waiting states of ACTS 57 to 59.

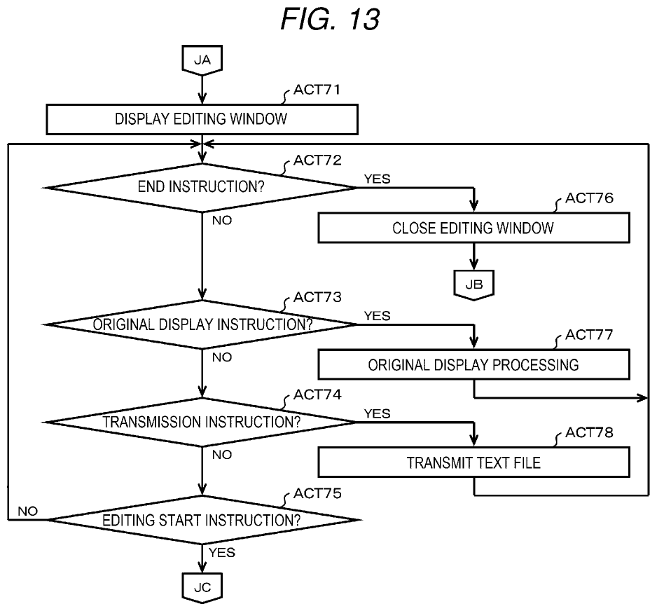

If an instruction to display the editing window is given as described above, the processor 21 gives YES in ACT 62 and proceeds to ACT 71 in FIG. 13.

In ACT 71, the processor 21 displays the editing window as a popup on the touch panel provided in the input and output unit 24. The editing window is a GUI screen to edit the text represented by the edited text file.

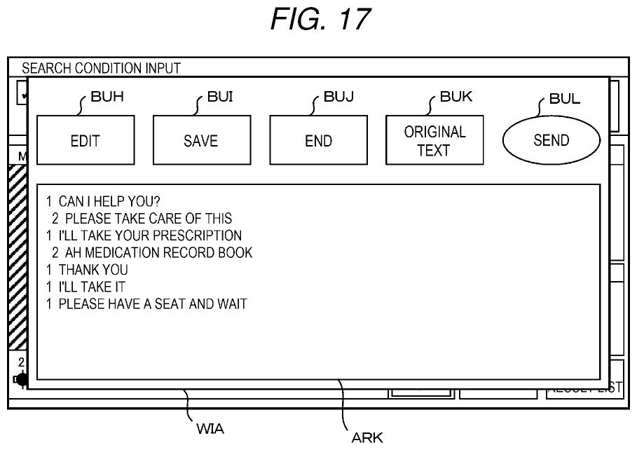

FIG. 17 shows an example of a screen where an editing window WIA is displayed as a popup.

The editing window WIA includes an area ARK and buttons BUH, BUI, BUJ, BUK, BUL. The area ARK shows the entirety of the text displayed in the area ARJ in the playback screen when the instruction to display the editing window is given. The button BUH is a soft key for the operator to give an instruction to start editing the text. The button BUI is a soft key for the operator to give an instruction to save the edited text. The button BUJ is a soft key for the operator to give an instruction to end the editing of the text. The button BUK is a soft key for the operator to give an instruction to display the original text before the editing, when the edited text is displayed in the area ARK. The button BUL is a soft key for the operator to give an instruction to transmit a text file representing the text displayed in the area ARK, to outside of the recording system 100.

After displaying the editing window in ACT 71 in FIG. 13, the processor 21 enters the waiting states of ACT 72, ACT 73, ACT 74, and ACT 75. In the waiting states of ACTS 72 to 75, the processor 21 waits for one of an end instruction, an original display instruction, a transmission instruction, and an editing start instruction to be given.

The checker checks the text displayed in area ARK. If the checker determines that there is no need to edit the text, the checker gives an instruction to end, for example, by a predetermined operation such as a tap on the button BUL. In response to this instruction, the processor 21 gives YES in ACT 72 and proceeds to ACT 76.

In ACT 76, the processor 21 closes the editing window WIA. The processor 21 then returns to the waiting states of ACT 62 and ACT 63 in FIG. 12.

The text displayed in the area ARK in the editing window WIA may have been edited in the past. If the checker wants to check the original before such editing, the checker gives an instruction to display the original, for example, by a predetermined operation such as a tap on the button BUK. In response to this instruction, the processor 21 gives YES in ACT 73 and proceeds to ACT 77.

In ACT 77, the processor 21 executes original display processing to display the original text. Specifically, for example, the processor 21 requests the recording and playback device 1 to provide the original text file. If the original text file is sent from the recording and playback device 1 in response to this request, the processor 21 displays a new window showing a text represented by the original text file, as a popup superimposed on the editing window WIA. After that, the processor 21 closes the foregoing window in response to an instruction to end the display of the original and then returns to the waiting states of ACTS 72 to 75.

At the recording and playback device 1, if the processor 11 receives the foregoing request for the original text file, the processor 11 gives YES in ACT 16 in FIG. 5 and proceeds to ACT 24.

In ACT 24, the processor 11 acquires an original text file that meets the request, from the text file group FIB, and transmits this original text file to the counter terminal 2. Specifically, for example, the processor 11 extracts an original text file specified by the original file path set in the field FBD of the service response data found in ACT 23, from the text file group FIB. The processor 11 then sends out this original text file addressed to the counter terminal 2, from the communication unit 14 to the communication network 200. If the edited text file transmitted in ACT 23 has never been edited even once, no original file path is set in the field FBD of the service response data found in ACT 23. In this case, the edited text file transmitted in ACT 23 is equivalent to the original text file. Therefore, the processor 21 transmits the edited text file transmitted in ACT 23, as the original text file. Alternatively, if no original file path is set, the processor 11 may notify that no original text file exists, instead of transmitting the original text file. After that, the processor 11 returns to the waiting states of ACTS 12 to 17.

If it is notified by the processor 11 that no original text file exists, as described above, the processor 21 of the counter terminal 2 causes the input and output unit 24 to perform a display or the like to cause the checker to recognize that the text already shown in the area ARK is the original, in ACT 77 in FIG. 13.

If the checker decides to transmit the text file representing the text displayed in the area ARK to outside of the recording system 100, the checker gives an instruction to transmit the text file, for example, by a predetermined operation such as a tap on the button BUL in the editing window. On receiving this instruction, the processor 21 gives YES in ACT 74 in FIG. 13 and proceeds to ACT 78.

In ACT 78, the processor 21 transmits the text file representing the text shown in the area ARK to a predetermined transmission destination. The processor 21 may accept the designation of a transmission destination and then transmit the text file to this designated transmission destination. After that, the processor 21 returns to the waiting states of ACTS 82 to 86.

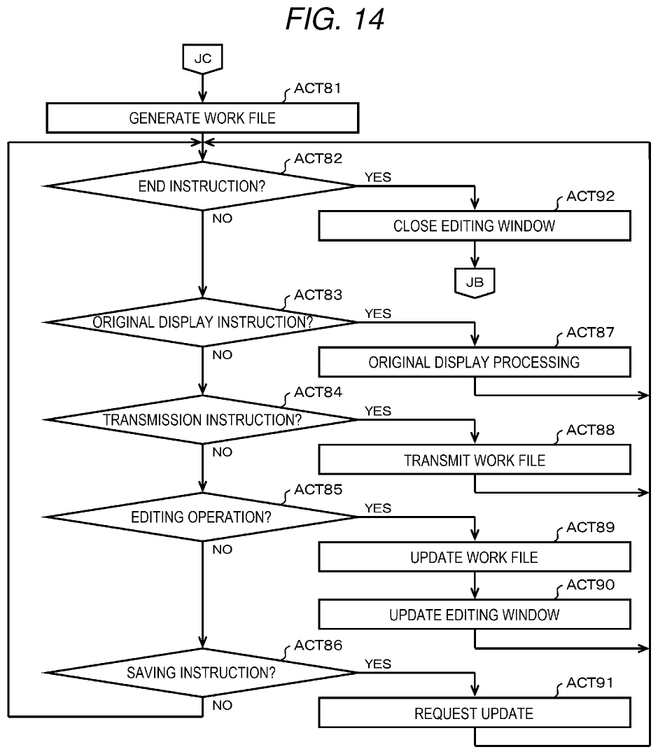

The checker checks the text shown in the area ARK. If the checker decides to edit the text, the checker gives an instruction to start editing by a predetermined operation such as a tap on the button BUH in the editing window WIA. On receiving this instruction, the processor 21 gives YES in ACT 75 and proceeds to ACT 81 in FIG. 14.

In ACT 81, the processor 21 copies the text file representing the text shown in the area ARK and thus generates a work file. After that, the processor 21 enters the waiting states of ACT 82, ACT 83, ACT 84, ACT 85, and ACT 86. In the waiting states of ACTS 82 to 86, the processor 21 waits for one of an end instruction, an original display instruction, a transmission instruction, an editing operation, and a saving instruction to be given.

If an original display instruction is given, the processor 21 gives YES in ACT 83, proceeds to ACT 87, and executes the original display processing as in ACT 77 in FIG. 13. After that, the processor 21 returns to the waiting states of ACTS 82 to 86.

If a transmission instruction is given, the processor 21 gives YES in ACT 84, proceeds to ACT 88, and transmits the text file as in ACT 78 in FIG. 13. However, in this case, the processor 21 uses the work file as the text file to be transmitted. After that, the processor 21 returns to the waiting states of ACTS 82 to 86.

If the checker is to edit the text shown in the area ARK, the checker performs a predetermined operation for the editing. This operation may be, for example, similar to an operation performed to edit a text in various existing information processing devices. For example, if the checker notices that “A medication record book” is shown by mistake instead of “Ah medication record book”, the checker performs an operation to replace “A” with “Ah”. In response to such an editing operation, the processor 21 gives YES in ACT 85 and proceeds to ACT 89. At this point, the processor 21 inputs an instruction to change the content of the text, as the editing operation That is, as the processor 21 executes the information processing based on the video search program PRC, the computer whose central unit is the processor 21 functions as an input unit.

In ACT 89, the processor 21 updates the work file in response to the operation.

In ACT 90, the processor 21 updates the editing window WIA in such a way that a text represented by the updated work file is shown in the area ARK. After that, the processor 21 returns to the waiting states of ACTS 82 to 86.

If the checker decides to save the result of the editing after finishing the editing, the checker gives an instruction to save the result of the editing, for example, by a predetermined operation such as a tap on the button BUI in the editing window WIA. If this saving instruction is given, the processor 21 gives YES in ACT 86 and proceeds to ACT 91.

In ACT 91, the processor 21 requests the recording and playback device 1 to update the edited text file, with a notification of the work file. After that, the processor 21 returns to the waiting states of ACTS 82 to 86.

At the recording and playback device 1, if the processor 11 receives the foregoing update request, the processor 11 gives YES in ACT 17 in FIG. 5 and proceeds to ACT 25.

In ACT 25, the processor 11 checks whether an original text file corresponding to the edited text data to be updated is already saved or not. For example, the processor 11 checks whether an original file path is set in the field FBD of the service response data found in ACT 23 or not. If the original file path is not set, the processor 11 gives NO, determining that the original text file is not saved, and then proceeds to ACT 26.

In ACT 26, the processor 11 saves a copy of the edited text file transmitted in ACT 23, in the text file group FIB, and defines this as the original text file. The processor 11 also sets the file path of this original text file as the original file path in the field FBD of the service response data found in ACT 23.

After finishing ACT 26, the processor 11 proceeds to ACT 27. If it is confirmed in ACT 25 that the original file path is set, the processor 11 gives YES, determining the original text file is saved. The processor 11 then skips ACT 26 and proceeds to ACT 27.

In ACT 27, the processor 11 overwrites the edited text file specified by the edited file path set in the field FBC of the service response found in ACT 23 with the work file which the processor 11 is notified of, and saves the work file, and thus rewrites the edited text file. After that, the processor 11 returns to the waiting states of ACT 12 to 17.

Thus, as the processor 11 executes the information processing based on the recording and playback program RPA, the computer whose central unit is the processor 11 functions as an update unit.

If the checker is to end the editing, the checker gives an instruction to end, for example, by a predetermined operation such as a tap on the button BUJ shown in the editing window WIA. In response to this instruction, the processor 21 gives YES in ACT 82 in FIG. 14 and proceeds to ACT 92.

In ACT 92, the processor 21 closes the editing window WIA. The processor 21 then returns to the waiting states of ACT 62 and ACT 63 in FIG. 12. At this point, if the work file is not saved even though the work file is updated, the editing performed up to this point by the checker is destroyed. Therefore, the checker may be notified that the editing that is not saved is invalid. The checker may then be allowed to designate whether to end the editing or not, and if the checker designates that the editing is not to end, the processor 21 may return to the waiting states of ACTS 82 to 86 in FIG. 14.

As described above, in the recording system 100, a video formed by capturing the state of a service response to patient and a text formed by the voice recognition of an utterance corresponding to the service response to patient are displayed, synchronized in time. Thus, the recording system 100 can facilitate the after-the-fact check of a face-to-face interaction between a plurality of people with a conversation.

In the recording system 100, the status of implementation of service responses to patient is recorded, and if one of the past service responses to patient is designated, the video and the text corresponding to the service response to patient are displayed. Therefore, the content of the utterance corresponding to a service response to patient that needs to be checked can be easily checked with the video.

In the recording system 100, a text is updated in response to an instruction given by a checker. Therefore, even if an error occurs in the voice recognition, the checker can easily correct an inadequacy in the text due to the error.

This embodiment can be implemented with various modifications as described below.

The playback screen may be generated by the processor 11 of the recording and playback device 1. In this case, the functions as the video display unit and the text display unit are implemented by the computer whose central unit is the processor 11.

The video file group FIA and the text file group FIB may be stored in the auxiliary memory unit 23 of the counter terminal 2 and the recording and playback processing may be performed by the processor 21 of the counter terminal 2. In this case, the functions as the video acquisition unit and the text acquisition unit are implemented by the computer whose central unit is the processor 21. Also, in this case, the update of the text file in order to reflect the result of editing should be performed by the processor 21. If the update of the text file is thus executed, the function as the update unit is implemented by the computer whose central unit is the processor 21.

At least one of the search processing and the voice recognition processing may be executed by an information processing device that is different from the counter terminal 2. That is, the search processing may be executed by an information processing device that is different from the counter terminal 2. Also, the voice recognition processing may be executed by an information processing device that is different from the counter terminal 2. In this case, the information processing device executing the search processing and the information processing device executing the voice recognition processing may be the same or different from each other.

The processor 11 may add only the original text file to the text file group FIB in ACT 20. If the editing processing is executed, the processor 11 may generate an edited text file that reflects the editing, and may add the generated edited text file to the text file group FIB.

A part or all of the functions implemented by the processors 11, 21 executing the information processing can be implemented by hardware executing information processing that is not based on a program, such as a logic circuit. Each of the foregoing functions can also be implemented by a combination of the foregoing hardware such as a logic circuit and software control.

While some embodiments of the present disclosure have been described, these embodiments are presented simply as examples and are not intended to limit the scope of the present disclosure. These novel embodiments can be implemented in various other forms and can include various omissions, replacements, and changes without departing from the spirit and scope of the present disclosure. These embodiments and modifications thereof are included in the spirit and scope of the present disclosure and also included in the scope of the claims and equivalents thereof.

What is claimed is:

1. A video playback system, comprising:
a video acquisition component configured to acquire video data representing a video captured over a predetermined image capture area;
a video memory component configured to store the video data acquired by the video acquisition component;
a text acquisition component configured to acquire text data generated by recognizing a voice collected around the image capture area;
a text memory component configured to store the text data acquired by the text acquisition component;
a video display component configured to display the video represented by the video data stored in the video memory component; and
a text display component configured to select text data generated by recognizing a voice collected around the image capture area at an image capture timing of the video displayed by the video display component, from among the text data stored in the text memory component, and display a text represented by the selected text data.

2. The video playback system according to claim 1, further comprising:
an image capture component configured to capture a video over the image capture area and generate video data, wherein
the video acquisition component acquires the video data generated by the image capture component.

3. The video playback system according to claim 1, further comprising:
a voice collection component configured to collect a voice around the image capture area, wherein
the text acquisition component acquires text data formed by recognizing the voice collected by the voice collection component.

4. The video playback system according to claim 1, further comprising:
an input component configured to input a change instruction to change a content of the text displayed by the text display component; and
an update component configured to update the text data stored in the text memory component in such a way that the content of the text displayed by the text display component is changed according to the change instruction inputted by the input component.

5. The video playback system according to claim 1, further comprising:
a server device comprising the video acquisition component, the video memory component, the text acquisition component, and the text memory component; and
a terminal device comprising the video display component and the text display component.

6. The video playback system according to claim 1, further comprising:
a video search component configured to search video data stored in the video memory component.

7. The video playback system according to claim 1, further comprising:
a voice recognition component configured to perform voice recognition processing on the voice collected around the image capture area.

8. The video playback system according to claim 1, further comprising:
a plurality of video display components each configured to display the video represented by the video data stored in the video memory component.

9. The video playback system according to claim 1, further comprising:
a plurality of text display components each configured to select text data generated by recognizing a voice collected around the image capture area at an image capture timing of the video displayed by the video display component and display a text represented by the selected text data.

10. The video playback system according to claim 1, further comprising:
an input and output component comprising a registration screen and a touch panel.

11. A video processing method, comprising:
acquiring video data representing a video captured over a predetermined image capture area;
storing the video data acquired;
acquiring text data generated by recognizing a voice collected around the image capture area;
storing the text data acquired;
displaying the video represented by the video data stored; and
selecting text data generated by recognizing a voice collected around the image capture area at an image capture timing of the video displayed, from among the text data stored, and displaying a text represented by the selected text data.

12. The video processing method according to claim 11, further comprising:
capturing a video over the image capture area and generating video data; and
acquiring the video data generated.

13. The video processing method according to claim 11, further comprising:
collecting a voice around the image capture area; and
acquiring text data formed by recognizing the voice collected.

14. The video processing method according to claim 11, further comprising:
inputting a change instruction to change a content of the text displayed; and
updating the text data stored in such a way that the content of the text displayed is changed according to the change instruction inputted.

15. The video processing method according to claim 11, wherein
a server device acquires video data, stores the data, acquires text data, and stores the text data; and
a terminal device displays the video and selects the text data.

16. The video processing method according to claim 11, further comprising:
searching video data stored.

17. The video processing method according to claim 11, further comprising:
performing voice recognition processing on the voice collected around the image capture area.

18. The video processing method according to claim 11, further comprising:
a plurality of video display components each displaying the video represented by the video data stored.

19. The video processing method according to claim 11, further comprising:
a plurality of text display components each selecting text data generated by recognizing a voice collected around the image capture area at an image capture timing of the video displayed and displaying a text represented by the selected text data.

20. The video processing method according to claim 11, further comprising:
registering a user.
